(19) United States
(12) Patent Application Publication
(10) Pub. No.: US 2024/0100345 A1
(43) Pub. Date: Mar. 28, 2024

(54) Methods for Inserting and Affixing an Implant in an Abdominal Wall Cavity by Using Laparoscopy

(71) Applicant: Galvani Bioelectronics Limited, Brentford, Middlesex (GB)

(72) Inventor: Eric Irwin, Collegeville, PA (US)

(21) Appl. No.: 18/267,686

(22) PCT Filed: Dec. 15, 2021
(86) PCT No.: PCT/EP2021/085858

§ 371 Date: Jun. 15, 2023

Related U.S. Application Data
(60) Provisional application No. 63/127,007, filed on Dec. 17, 2020.

(51) Int. Cl. A61N 1/375; A61B 17/04; A61B 17/10; A61B 17/34; A61N 1/05

(52) U.S. Cl. CPC A61N 1/37518; A61B 17/0469; A61B 17/10; A61B 17/3468; A61N 1/0507; A61B 2017/320044

(57) ABSTRACT

A method of inserting and affixing an implant by making an incision in an abdominal wall is disclosed. A subcutaneous pocket is formed between a first layer of the abdominal wall and a second layer of the abdominal wall, such that the pocket is at least 2 centimeters from the costal margin. An implant is placed into the subcutaneous pocked and affixed within the subcutaneous pocket in the abdominal wall.

### CROSS-REFERENCE TO RELATED APPLICATIONS

This application claims priority to U.S. Provisional Application 63/127,007, filed Dec. 17, 2020, the contents of which are incorporated by reference herein in its entirety.

### TECHNICAL FIELD

The disclosed implementations relate generally to a method for inserting and affixing an implant.

### BACKGROUND

Medical devices exist that deliver stimulation to therapeutic targets within a body of a patient. For example, implants, such as pacemakers, deliver stimulation to one or more regions of a heart. Often these medical decides draw power from a power supply to function. Some components of implants are implanted subcutaneously within the body of the patient.

Current technologies and methods have several challenges. Technologies & methodologies for implanting components of an implant, for example, a power supply, using traditional open techniques can create a large pocket size. Larger pocket sizes allow movement of the implant, which can cause loss of connection, diminishment of battery for re-charging, and potential tissue damage. Further, a mismatch between a pocket and an implant, or a component of an implant, may predispose to the formation of seromas, which may lead to an increased risk of wound complications, including infection. Therefore, a method of inserting an implant and affixing is needed such that the implant, or a component of the implant, is secured into a pocket that is appropriately sized for the implant such that movement within the pocket is minimized, thus offering the potential for reduced loss of connection, diminishment of battery, or tissue damage.

### SUMMARY

Accordingly, implementations of the present invention are directed to methods for inserting and affixing an implant into an abdominal wall cavity. In accordance with some implementations, a method of inserting an implant includes making a first incision in the abdominal wall. The method further includes forming a pocket between a first layer of the abdominal wall and a second layer of the abdominal wall, such that the pocket is kept at least 2 cm from the costal margin, such that the first incision defines an opening into the pocket. The implant is then inserted through the opening and into the pocket. Once the implant is inserted, the method includes positioning the implant into the pocket. The implant may be affixed in the subcutaneous tissue with sutures, by providing puncture wounds, for example two puncture wounds, through the abdominal wall musculature at the lateral extent of the pocket. In another embodiment, the implant may be affixed in the subcutaneous tissue with sutures by providing more than two puncture wounds through the abdominal wall musculature at the lateral extent of the pocket. The puncture wounds are made with laparoscopic visualization for monitoring. The sutures may be passed through the abdominal wall musculature into the abdominal cavity. The puncture wounds may be at least 2 cm from the costal margin. The method further comprises closing the opening such that the implant is secured within the pocket. Another method of inserting and affixing an implant is described in Patent Application PCT/GB2018/053767 filed Dec. 21, 2018, entitled “Systems and Methods Configured to Insert an Implant in an Abdominal Wall Cavity,” which is incorporated by reference in its entirety. The implant may be an implantable pulse generator (IPG), and an example of such IPG is described in Patent Application PCT/US2019/052403 filed on 23 Sep. 2019, entitled “Implantable Pulse Generator with Suture Holes, Methods for Implanting the same, and Encapsulation of External Components in Active Implantable Medical Devices”, which is incorporated by reference in its entirety.

Both the foregoing general description and the following detailed description are exemplary and explanatory and are intended to provide further explanation of the invention as claimed.

### BRIEF DESCRIPTION OF THE DRAWINGS

For a better understanding of the aforementioned systems, and methods, as well as additional systems and methods, reference should be made to the Detailed Description of Implementations below, in conjunction with the following drawings in which like reference numerals refer to corresponding parts throughout the figures.

FIG. 1 is a frontal view of surgical incisions for implant positioning;

FIG. 2A is a view of layers of the abdominal wall;

FIG. 2B is a view of a pocket being formed in the area illustrated in FIG. 2A;

FIG. 3 illustrates the pocket from FIGS. 2A-2B being prepared for suturing, with suture placement, the inset showing the sutures in place in the fascia;

FIG. 4 illustrates a lead being introduced through the abdominal wall;

FIG. 5 illustrates positioning of an implant into the pocket created in FIGS. 2A-2B.

FIG. 6 illustrates an implant and sutures and stitches affixing an implant according to some example embodiments.

Reference will now be made to implementations, examples of which are illustrated in the accompanying drawings. In the following description, numerous specific details are set forth in order to provide a thorough understanding of the present invention. However, it will be apparent to one of ordinary skill in the art that the present invention may be practiced without requiring these specific details.

### DETAILED DESCRIPTION OF IMPLEMENTATIONS

Aspects of the disclosure will now be described in detail with reference to the drawings, wherein like reference numbers refer to like elements throughout, unless specified otherwise. Certain terminology is used in the following description for convenience only and is not limiting. The term “plurality,” as used herein, means more than one. The terms “a portion” and “at least a portion” of a structure include the entirety of the structure. An “implant” may be a neuromodulation device, a neuromodulation system that comprises one or more neuromodulation devices, an implantable pulse generator (IPG), a device that sends electrical signals to specific targets, one component of a system that delivers electrical signals to a target, for example the target may be a blood vessel, a lymph node, a nerve (e.g. a vagal nerve or splenic nerve), tissue (e.g. vascular tissues or perivasculature tissues) or organ (e.g. spleen). Certain features of the disclosure, which are described herein in the context of separate embodiments, may also be provided in combination in a single embodiment. Conversely, various features of the disclosure that are described in the context of a single embodiment may also be provided separately or in any subcombination.

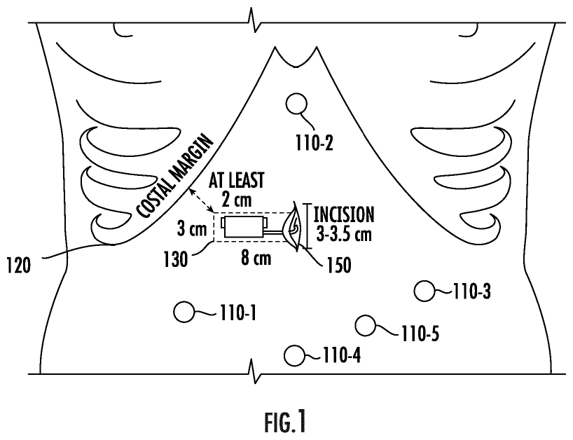

FIG. 1 illustrates is a frontal view of surgical incisions and a plurality of incisions for placement of an introduction tool for an implant positioning. The introductory tool may be a trocar. The introduction tool may be a laparoscopic trocar. In other embodiments, the introduction tool may be a hand assisted laparoscopy port. In other embodiments, the introduction tool may be through a robotic port. In further embodiments, the introduction tool is any tool that allows introduction of medical equipment useful in a minimally invasive surgical procedure (e.g. laparoscopic surgery) or the removal of specimens. An incision 150 may be made in an upper abdominal portion of the patient in the midline of the abdominal wall to gain access to the subcutaneous tissues for forming a pocket into which an implant may be introduced and affixed to the abdominal wall, such that the pocket, extends laterally, for example, extends 8 centimeters (cm) laterally, and is at least 2 centimeters from the costal margin 120. In some embodiments, the incision 150 is between 2.0 centimeters and 4.0 centimeters in length. In some embodiments, the incision is between 3.0 and 3.5 centimeters in length. In an additional embodiment, an incision may be made in the lower abdominal portion of the patient, for example, in the midline of the abdominal wall to gain access to the subcutaneous tissues for forming a pocket into which an implant may be introduced and affixed to the abdominal wall, such that the pocket, extends laterally, for example, extends 8 centimeters (cm) laterally, and is at least 2 centimeters. In some embodiments, the incision 150 is between 2.0 centimeters and 4.0 centimeters in length. In some embodiments, the incision is between 3.0 and 3.5 centimeters in length.

The incision may be located in the midline such that the distance from the midline to the costal margin 120 is 10 cm or more. The incision may be carried down to the level of the fascia and a plane may be developed between the fascia and the subcutaneous tissues. The implant may be inserted through the midline opening into the pocket and positioned using sutures. Sutures may be placed by one or more instruments introduced via one or more introduction tools, for example, one or more trocars or hand assisted laparoscopic ports. Sutures may also be placed by methods and techniques used for introducing instruments used in minimally invasive procedures. For example, robotics may be used to manipulate the sutures. The grasper 420 may be a laparoscopic grasper or a robotic grasper. The opening is closed such that the implant is secured within the pocket.

In some embodiments, the two incisions that are most lateral (i.e., 110-1 and 110-3), are formed to accommodate introduction tools and may be used to allow the introduction of graspers and retractors and other laparoscopic or robotic instruments. In some embodiments, one or more 5 millimeter trocars may be used. In other embodiments, one or more 10 millimeter trocars may be used. In additional embodiments, one or more 12 millimeter trocars may be used. In further embodiments, one or more 15 millimeter trocars may be used. In some embodiments, incision 110-1 can be used for an introduction tool that allows the surgeon to provide counter traction, for example for grasping the suture during suture placement (e.g. transfascial suture placement).

In some embodiments, there is an incision made at the umbilicus that is formed to accommodate an umbilical introduction tool, for example an umbilical trocar. In some embodiments, incision 110-4 is used to accommodate a trocar that is 10 millimeters or 12 millimeters or between 10 millimeters and 12 millimeters. In some embodiments, incision 110-4 is used for laparoscopy and other instruments that may be needed, for example, other 12 millimeter instruments.

In additional embodiment, there is an incision cranial to the umbilicus, for example in the midline, that is formed to accommodate an introduction tool, for example a trocar, for example a laparoscopic trocar or a robotic trocar. In some embodiments, incision 110-4 is used to accommodate a trocar that is 10 millimeters or 12 millimeters or between 10 millimeters and 12 millimeters. In some embodiments, incision 110-4 is used for laparoscopy and other instruments that may be needed, for example, other 12 millimeter instruments.

In some embodiments, incision 110-5 may be used to accommodate an introduction for a lead and for introducing the lead of the implant. In some embodiments, incision 110-5 is made to accommodate a trocar, for example a 15 millimeter trocar. In some embodiments, the trocar is an oblong trocar for implant 130 insertion. In some embodiments, incision 110-5 is used by the surgeon while preparing a site for a neural interface, inserting the neural interface, placing the neural interface around a nerve and/or artery, and/or suturing. In one embodiment, the neural interface, may be a cuff neural interface comprising a cuff portion. In other embodiments, the neural interface system may be paddle type, wrap-type, linear rod-type or any other type of electrode appropriate for the specific therapy.

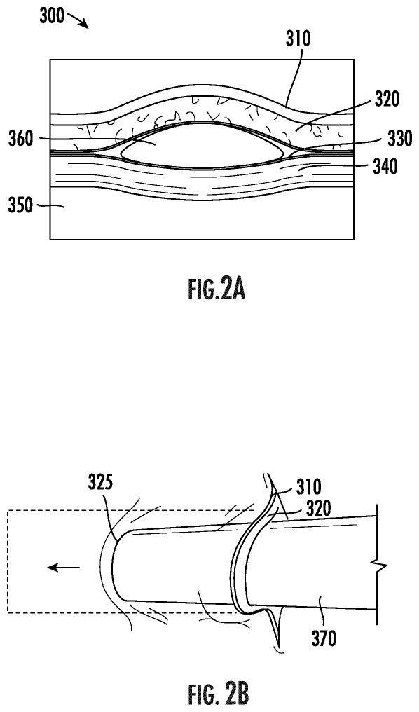

FIG. 2A illustrates a view of layers of the abdominal wall for the pocket 360 creation. The pocket 360 is created in the layers between the skin surface 310 and the abdominal cavity 350. In some embodiments, the pocket 360 is fashioned between the subcutaneous tissues 320 and the rectus abdominus muscle 340. In some embodiments, the pocket 360 is positioned between the subcutaneous tissues 320 and the rectus sheath 330. In some embodiments, the pocket 360 extends onto the oblique musculature.

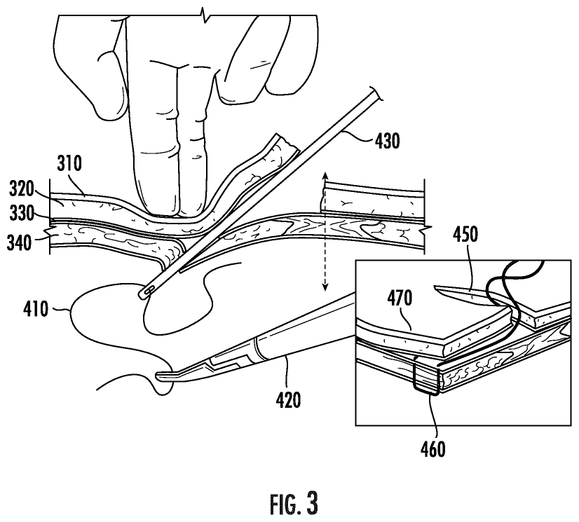

FIG. 2B is a view of the pocket 360 being formed in the area illustrated in FIG. 3A between the subcutaneous tissues 320 and rectus sheath 330. The pocket 360 is fashioned starting at the midline incision 150 (FIG. 1) and extending for about 8 centimeters (for example, from 7.5 centimeters to 8.5 centimeters), for an implant with the dimensions of 4.5 cm long, 2 cm wide and 0.7 cm thick with the lead exiting the implant medially and suture tabs located at the ends of the implant.

In systems where the implant does not include fixation tab(s) the pocket should be oversized approximately 0.5 cm (for example 0.25 cm to 0.75 cm) except on the side where the lead exits the implant where it may be necessary to include an additional 1.5 cm-2 cm to avoid kinking of the lead body as it exits the implant. In other embodiments, the pocket may be approximately 2 cm longer (for example, 1 cm to 3 cm longer) than the length required for the implant and lead as it exits the implant. In another embodiment, if the lead implant is 4 cm by 4 cm, the pocket may extend approximately 0.5 cm, for example 4 centimeters to 6 centimeters.

In a further embodiment, if the implant thickness is a centimeter or less, the pocket may be oversized by 0.5 cm, i.e. 0.5 cm in addition to the thickness of the implant. Alternatively, if the implant thickness exceeds 1 centimeter, the pocket may be oversized by more than 0.5 cm, i.e. more than 0.5 cm in addition to the thickness of the implant. In a further embodiment, the width of the pocket may be oversized approximately 1 centimeter beyond the width of the implant, for example 0.5 centimeter to 1.5 centers beyond the width of the implant. The width of the pocket may be approximately 3 centimeters, for example 2.5 centimeters-3.5 centimeters in width. In cases where the fixation system is different from that found in this system the pocket size should accommodate for this. A benefit of using this technique is to minimize any oversizing of the pocket relative to the IPG size.

In some embodiments, the pocket 360 is created by using a blunt dissection 370. In some embodiments, the blunt edge 352 slides between the subcutaneous tissues 320 and the rectus sheath 330 thereby allowing the subcutaneous tissues 320 to release from the rectus sheath 330 allowing for a pocket 360. In some embodiments, the pocket 360 is created by using a Deaver retractor, ribbon retractor, malleable retractor, balloon dissector, or any suitable combination thereof. In some embodiments, the pocket 360 can be enlarged by using any of the above described tools. In some embodiments the Deaver retractor, ribbon retractor, and malleable retractor are about 3.5 centimeters wide. In some embodiments, the Deaver retractor, ribbon retractor, and malleable retractor are greater than 3.5 centimeters wide. In some embodiments, the Deaver retractor, ribbon retractor, and malleable retractor are smaller than 3.5 centimeters wide. In a preferred embodiment, the Deaver retractor, ribbon retractor, and malleable retractor are 3 centimeters wide.

After pocket creation, the pocket may be inspected to confirm hemostasis. In some embodiments, the pocket may be packed with a sponge that has been moistened with saline. In some embodiments, the pocket may be packed with a sponge that has been moistened with a dilute solution of adrenaline in saline. In some embodiments, if locally applied pressure and/or the judicious use of vasoconstrictive agents are not successful, topic hemostatic agents such as thrombin or Tisseel may be considered.

FIG. 3 illustrates the pocket from FIGS. 2A-2B being prepared for suturing. Once hemostasis is confirmed, the preparations are made for the fixation of an implant within the pocket. In some embodiments the implant is an implantable pulse generator (herein referred to as IPG and/or implant). In some embodiments, a suture passer 430 is used to place a suture comprising a suture material 410 at the deep end of the pocket. In some embodiments, the suture passer 430 is AN ENDO CLOSE™ or a CARTER-THOMASON® CloseSure system.

In some embodiments, the suture is a U-stitch 460. In some embodiments, two transfascial sutures may be placed, one at each corner of the suture tab, or the suture may be a T-bar type system. In some embodiments, the suture 410 is any suitable permanent suture material deemed appropriate by the implanting surgeon. In some embodiments, the suture material is a monofilamentous suture (e.g. a synthetic monofilamentous material), for example monofilamentous polypropylene or monofilamentous nylon. For example, the suture material may be PROLENE® (a sterile, synthetic, non-absorbable, monofilament, surgical suture made from an isotactic crystalline stereoisomer of polypropylene). In other embodiments, the suture material may be a braided material, for example a braided Nylon. An example of a braided Nylon is NUROLON® (a braided suture composed of the long chain aliphatic polymer Nylon 6,6).

In some embodiments, the suture material 410 is grasped with the suture passer 430, which under direct laparoscopic visualization is then advanced obliquely through the fascia in the upper most distal, farthest from the midline incision, corner of the pocket. The suture material 410 is then grasped with a pair of graspers 420 from within the abdominal cavity and the suture passer 430 withdrawn. In another embodiment, the suture material 410 is introduced into the tabs on the IPG and ends of the sutures are passed through the abdominal wall from the subcutaneous pocket into the abdominal cavity, where the suture is secured with intracorporal knot tying. In some embodiments, the suture material 410 is a synthetic monofilament material. In further embodiments, the suture material is polypropylene, for example 2-0 PROLENE®. In some embodiments, the material is nylon. In some embodiments, the material is a braided nylon. In another embodiment, the suture material is chosen by the implanting surgeon.

In some embodiments, the suture passer 430 is reintroduced through the fascia in the lower most distal corner of the pocket obliquely through the abdominal wall cavity. The reintroduction of the suture passer 430 is accomplished under direct laparoscopic visualization.

In some embodiments, the suture is brought out of the pocket through the incision and the suture material 410 is clamped to maintain proper cranial to caudal alignment.

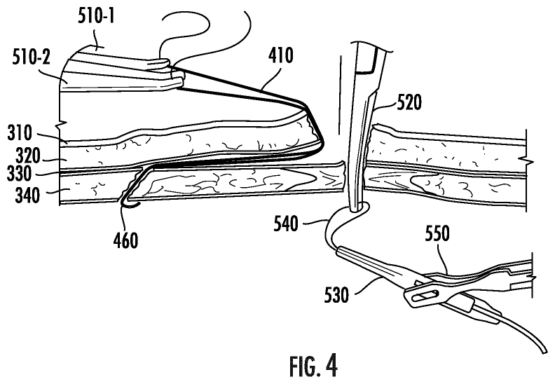

FIG. 4 illustrates a lead being introduced through the abdominal wall. In some embodiments, once the midline fascia is identified, a clamp is advanced perpendicularly through the linea alba into the abdominal cavity. In some embodiments, the introduction is done under direct laparoscopic vision. In other embodiments, the incision may be extended and the lead cap may be grasped under direct vision.

In some embodiments, the proximal end of the lead cap 530 with a suture loop 540 is identified and brought to the clamp 520. In some embodiments, the lead cap 530 may be grasped by a tool 550, wherein the tool is a clamp, grasper, or any suitable tool thereof. The suture loop 540 of the lead cap 530 is grasped by the clamp 520 and the lead brought through the abdominal wall into the incision used to create the subcutaneous pocket. In some embodiments, the lead body can be grasped with an atraumatic grasper and drawn into the abdominal cavity.

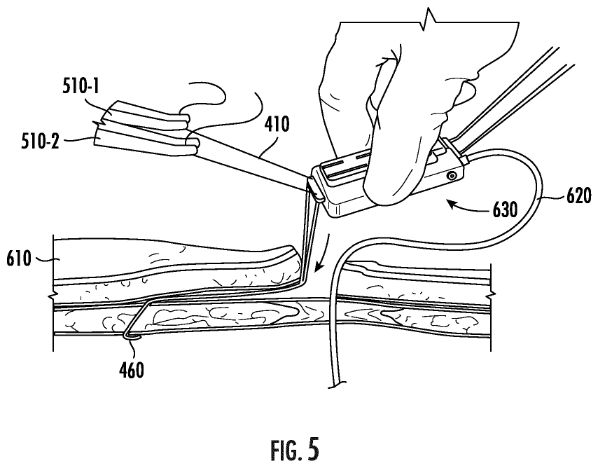

FIG. 5 illustrates the positioning of an implant into the pocket created in FIGS. 2A-2B. In some embodiments, the implant is oriented such that an antenna is facing towards the skin. In some embodiments, the antenna may be provided for communication and/or energy transmission. In some embodiments, the antenna is a Bluetooth antenna. In some embodiments, the antenna may be an RF antenna. In some embodiments, two sutures are placed in suture holes located at the end of the implant. In some embodiments, the implant is attached to a lead 620. In some embodiments, the needle is passed from ventral to dorsal so that it is in a position for placement of the sutures into the fascia. By doing so, the suture 460 is placed as shown in FIG. 5. In some embodiments, the sutures are then clamped to maintain proper orientation. In some embodiments, one or more clamps 510-1, 510-2 may be used to position the suture once the U-stitch 460 has been placed for proper alignment of the implant into the cavity. In some embodiments, the skin is covered with an antimicrobial drape 610.

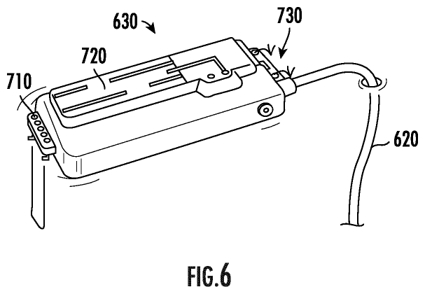

FIG. 6 illustrates an implant and sutures for affixing the implant according to some example embodiments. One or more sutures 730, for example, medial fascial sutures, may be used to affix the implant to the patient's body. In one embodiment two or more sutures 730, for example, medial fascial sutures, may be used to affix the implant to the patient's body. The one or more sutures may be placed through one or more openings (or suture engagement holes) in the body of the implant. The one or more openings may be provided in a suture engagement component portion of the implant body, for example when the implant is an implantable pulse generator (IPG).

The two or more sutures may be placed through one or more openings in the body of the implant. The two or more sutures may be placed through two or more openings in the body of the implant. In one embodiment, the one or more openings are on the side of the implant where the lead is attached. In one embodiment, the two or more openings are on the side of the implant where the lead is attached. In another embodiment, sutures may be affixed on any side of the implant.

In one embodiment, one or more sutures are placed through the anterior rectus sheath. In another embodiment, two or more sutures are placed through the anterior rectus sheath. A suture 710, for example a transfascial U-stitch, may also be used to affix the implant to the patient's body. In one embodiment, the suture may pass through the anterior rectus sheath, rectus muscles and posterior rectus muscle sheath. In another embodiment, the suture may be placed through an opening in the body of implant. In another embodiment, the suture may be placed through an opening of the implant opposite the lead connection. In some embodiments, the implant may comprise a power source, an antenna, or both. The power source may include a rechargeable battery configured to receive power from a source remote from the implant.

The antenna can be configured to transmit data, receive data, or both. According to one aspect of the disclosure, the antenna can be configured to transmit data about the implant, for example an amount of power remaining in the power supply, to a data receiver that is remote from the implant. For example, the antenna can be configured such that when the implant is positioned within the pocket, the antenna can transmit or receive data from a data receiver or data source located outside of the patient's body. In some embodiments, a plurality of antennae may be provided.

In some embodiments, the implant comprises a Bluetooth antenna 720, wherein the antenna is facing the skin. In some embodiments, the suture 710 is on the opposite end of the lead 620. In some embodiments, the sutures 730 are placed on the opposite end of the suture 710 and placed such that damage to the lead is avoided. In some embodiments, the suture 710 is on the lateral end of the pocket and the sutures 730 are on the medial end of the pocket.

In some embodiments, the laparoscope is reintroduced and the transfascial suture is inspected.

The implant may be positioned such that the proximal end of the pocket is opened by applying a gentle traction on the skin edges. In some embodiments, the suture is placed at the distal end of the pocket. In some embodiments, the suture is placed under tension until the redundancy has vanished intra-abdominally using the laparoscope. In some embodiments, the implant is then introduced into the pocket using a parachute technique. In some embodiments, the transfascial U-stitch is then tied while ensuring adequate tension using the laparoscope. In some embodiments, another suture is tied. In some embodiments, this is accomplished by using the attached needles to take passes through the fascia at the location that assures fixation of the implant without angulation between the connector side of the implant and the location in the linea alba where the lead body is introduced into the abdominal cavity. The sutures are tied under direct visualization. A laparoscope may be used to confirm that the U-stitch is tied.

Subsequently, the sutures are tied to secure the implant in place in the pocket. If stability of the implant is deemed satisfactory the sutures are then cut. A laparoscope may be used to confirm that all redundant suture material is removed from the abdomen.

The terminology used in the description of the invention herein is for the purpose of describing particular implementations only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The foregoing description, for purpose of explanation, has been described with reference to specific implementations. However, the illustrative discussions above are not intended to be exhaustive or to limit the invention to the precise forms disclosed. Many modifications and variations are possible in view of the above teachings. The implementations were chosen and described in order to best explain the principles of the invention and its practical applications, to thereby enable others skilled in the art to best utilize the invention and various implementations with various modifications as are suited to the particular use contemplated.

1. A method of inserting and affixing an implant, the method comprising the steps of:
making a first incision in an abdominal wall;
forming a subcutaneous pocket between a first layer of the abdominal wall and a second layer of the abdominal wall, such that the pocket is at least 2 centimeters from the costal margin, and such that the first incision defines an opening into the pocket;
inserting an implant through the opening and into the pocket;
positioning the implant into the pocket;
affixing the implant within the subcutaneous pocket; and
closing the opening such that the implant is secured within the pocket.

2. The method of claim 1, further comprising creating a second incision for a first introduction tool.

3. The method of claim 1, the method further comprising creating additional incisions for one or more introduction tools.

4. The method of claim 3, wherein the method further comprises forming a first incision and four additional incisions for one or more introduction tools.

5. (canceled)

6. The method of claim 3, wherein each of the incisions for the one or more introduction tools accommodates at least a 10 mm trocar.

7. The method of claim 6, wherein the one or more introduction tools comprises an umbilical introduction tool, wherein the umbilical introduction tool is a 15 millimeter trocar.

8. The method of claim 3, wherein an introduction tool in the one or more introduction tools creates a port to accommodate a lead.

9. (canceled)

10. The method of claim 1, wherein the subcutaneous pocket is between subcutaneous tissue and the rectus sheath

11. The method of any one of claim 1, wherein the subcutaneous pocket is 7.5 centimeters to 8.5 centimeters in length.

12. A method of creating a pocket for securing a surgical implant between subcutaneous tissue and rectus abdominus muscle, the method comprising the steps of:
making a first incision in an abdominal wall;
inserting a surgical instrument into the first incision; and
using the surgical instrument to extend the pocket from the first incision, wherein the pocket is at least 2 centimeters from the costal margin.

13. The method of claim 12, wherein inserting the surgical instrument further comprises developing the pocket using blunt dissection.

14. The method of claim 12, wherein the pocket is 7.5 centimeters to 8.5 centimeters in length.

15. The method of claim 12, wherein the pocket is 2.5 centimeters to 3.5 centimeters in width.

16. The method of claim 12, further comprising inserting and affixing an implant in the pocket.

17. A method of inserting an implant, the method comprising the steps of:
making a first incision in an abdominal wall;
forming a pocket between subcutaneous tissue and the rectus sheath by using a surgical instrument to extend the pocket from the first incision, such that the first incision defines an opening into the pocket;
inserting an implant through the opening and into the pocket;
positioning the implant into the pocket; and
closing the opening such that the implant is secured by at least one suture within the pocket.

18. The method of claim 17, wherein the pocket is at least 2 centimeters from the costal margin.

19. (canceled)

20. The method of claim 17, wherein the closing further comprises grasping the suture with a suture passer through one of the introduction tools, wherein the suture passer is visualized under laparoscopic visualization.

21. The method of claim 17, wherein the closing further comprises advancing the suture passer through the fascia in an upper distal corner of the pocket.

22. The method of claim 17, further comprising clamping the suture to maintain proper cranial to caudal alignment.

23. The method of claim 17, wherein the suture is selected from the group of: a U-stitch and a staple.

24. (canceled)
